(19) United States
(12) Patent Application Publication
(10) Pub. No.: US 2025/0248378 A1
(43) Pub. Date: Aug. 7, 2025

(54) TRANSGENIC ANIMALS FOR MERCURY BIOREMEDIATION

(71) Applicant: MACQUARIE UNIVERSITY, North Ryde, New South Wales (AU)

(72) Inventors: Maciej MASELKO, North Ryde, New South Wales (AU); Kaitlyn TEPPER, North Ryde, New South Wales (AU)

(21) Appl. No.: 18/852,664

(22) PCT Filed: Mar. 31, 2023
(86) PCT No.: PCT/AU2023/050248

§ 371 Date: Sep. 30, 2024

(30) Foreign Application Priority Data
Apr. 1, 2022 (AU) 2022900856

(51) Int. Cl. A01K 67/68; A62D 3/02; C12N 9/02; C12N 9/88; C12N 15/52

(52) U.S. Cl. CPC A01K 67/68; A62D 3/02; C12N 9/0091; C12N 9/88; C12N 15/52; A01K 2217/052; A01K 2227/706; C12Y 116/01001; C12Y 499/01002

(57) ABSTRACT

There is provided a transgenic animal comprising heterologous nucleic acid encoding a bacterial organomercurial lyase and/or a bacterial mercuric reductase, wherein the transgenic animal expresses the bacterial organomercurial lyase and/or a bacterial mercuric reductase to reduce the toxicity of a mercury compound.

### TECHNICAL FIELD

The technology relates to transgenic animals such as herbivores, fish, and insects, expressing either or both bacterial enzymes MerA and MerB to allow the animals to reduce methylmercury and/or inorganic mercury to elemental Hg(0). The transgenic animals can be used to graze pastures on mercury contaminated land, disrupt biomagnification of mercury in food chains, and treat organic wastes contaminated with mercury.

### CROSS REFERENCE TO RELATED APPLICATION

This application claims the benefit of Australian Provisional Application No. 2022900856 filed 1 Apr. 2022, the entire content of which is incorporated by reference herein.

### BACKGROUND

Mercury is a highly toxic heavy metal that is naturally occurring in the earth's crust. It is released into the biosphere through geogenic sources such as volcanies, rock weathering, and hydrothermal vents, and through human industrial activities such as mining, burning biomass and fossils fuels, metal production, cement production, vinyl-chloride production, and from the waste sector. It is estimated that human activities have increased the mercury concentration in the biosphere 450% over natural levels.

The toxicity, solubility, and volatility of mercury is influenced by its oxidation state and the specific compounds that contain it. Elemental mercury (Hg) is volatile and can be carried around in the earth's atmosphere, where it is oxidised through abiotic processes into inorganic mercury (Hg²⁺) and deposited into water, soil, and plants. Humans are typically exposed to elemental and inorganic mercury through occupational exposure. Furthermore, crops nearby industrial sources can accumulate inorganic mercury above the provisional tolerable weekly intake (PWTL), where its consumption can damage the renal, digestive, immune, and nervous systems. In the environment, inorganic mercury is utilised by iron- and sulphur-reducing bacteria for their metabolism and growth, generating methylmercury. Methylmercury is the most toxic form of mercury that readily bioaccumulates in food chains, particularly in marine environments. For humans, the highest source of mercury exposure is as methylmercury, typically from seafood in our diet. Accumulative exposure to methylmercury can impair brain development, lead to neurodegeneration, and impact fertility.

Currently mercury exposure is managed through legislation to prevent the release of mercury into the environment. The Minamata Convention (2013) has been ratified across many countries across the globe to control mercury sale and trade; reduce the use, emission, and release of mercury; raise public awareness; and build the necessary institutional capacity to reduce mercury emissions to the environment. However, reductions in mercury emissions and resulting declines in atmospheric concentrations may take time to show up as reductions of mercury concentrations in biota. For some time to come, methylmercury will continue to be produced from the legacy mercury previously deposited into soils, sediments, and aquatic systems. Local governments also legislate industrial and occupational discharge limits and safe disposal procedures, and limits for food and water supplies. Governments may provide consumers with dietary guidelines for seafood consumption and provide warnings to recreational fishers about contaminated areas that may contain fish with dangerously high levels of methylmercury.

To date, no technology has been implemented that can clean up mercury directly from within the food chain. Furthermore, regulatory changes are driving a demand for scalable and economic methods for treating mercury contaminated sites and removing it from feed and waste without using incineration or landfill sites. The mercury content of organic wastes, particularly from municipal biosolids and fisheries waste, may result in contamination and preclude its use as value-added fertilizers or animal feed.

The bacterial MerB (organomercurial lyase) and MerA (mercuric reductase) enzymes are capable of detoxifying mercury compounds including ionic mercury, Hg(II), methylmercury, and phenylmercury as shown in Reactions 1 and 2 (see below). In particular MerB transforms methylmercury to the less toxic ionic mercury Hg(II). MerA reduces Hg(II) to the least toxic metallic mercury, Hg(0).

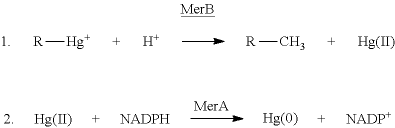

While certain microbial populations that naturally express MerA and/or MerB can detoxify mercury compounds in the environment such microbial populations are difficult to reliably use in practice as they face significant competition from native microbes at contaminated sites and cannot access mercury compounds present within plant or animal tissues.

Another approach to clean up mercury has been phytoremediation, the use of plants for bioremediation. It is known that when MerB and MerA enzymes are expressed in transgenic plants in laboratory conditions, the coupled reaction transforms and detoxifies methylmercury to volatile Hg(0). Alternatively, other studies have generated transgenic plants that can instead bioaccumulate and sequester mercury, where the contaminated plants can be harvested and disposed in hazardous waste.

However, transgenic plants cannot access mercury where it is most toxic and sequestered in the food chain, or where the contaminated material is organic waste. Furthermore, the volatilisation of mercury by plants may be problematic as mercury is dispersed back into the environment where it may re-enter the food chain and cause harm. Plants that instead bioaccumulate and sequester mercury over the course of months to years, may be inadvertently eaten by native herbivorous animals and also enter the food chain. Phytoremediation has major problems with scalability. Generating field adapted transgenic plants for each contaminated site may not be scalable in the variable climatic conditions at different contaminated sites. Transgene biocontainment may be problematic in areas that may contain related local plants. Utilizing phytoremediation also requires laboriously removing all the native plants from many contaminated sites, waiting months to years for a plant to grow, and potentially putting up fences to stop native herbivores from eating the contaminated transgenic plants.

Thus, there is a need for transgenic animals for the detoxification of mercury within food chains and in organic waste. Transgenic animals offer many unique benefits to mercury bioremediation. They are able to access mercury sequestered within other organisms via their digestive processes. They do not face fierce competition from microbes in the environment or in the microbiome. Large quantities of contaminated food can be consumed by the animal to enable sufficient enzyme expression for bioremediation. They can tolerate broad environmental conditions with high metabolic rates. Domestic herbivores can rapidly process large quantities of contaminated plant material with minimal human intervention and can subsequently be transported to other contaminated sites. Farmed animals such as fish can clean up contaminated feed and provide economical protein with all the nutritional benefits of fish but without methylmercury. Insects can be used to increase the value of contaminated organic waste streams; where the volatilized mercury can be trapped and stored away from the biosphere, the remaining organic waste could be utilised as soil amendment, and the insects can be used as an economical source of protein. Animals also have effective transgene biocontainment strategies including established desexing techniques, genetic sterilisation/incompatibility, and physical containment in the case of farmed animals or insects.

### SUMMARY

In a first aspect, there is provided a transgenic animal comprising heterologous nucleic acid encoding a bacterial organomercurial lyase and/or a bacterial mercuric reductase, wherein the transgenic animal expresses the bacterial organomercurial lyase and/or the bacterial mercuric reductase to reduce the toxicity of a mercury compound.

In one embodiment the heterologous nucleic acid encodes the bacterial organomercurial lyase and the mercury compound is an organomercurial compound. In another embodiment the heterologous nucleic acid encodes the bacterial mercuric reductase and the mercury compound is an inorganic mercury compound. In a further embodiment the heterologous nucleic acid encodes the bacterial organomercurial lyase and the bacterial mercuric reductase and the mercury compound is an organomercurial compound and/or inorganic mercury compound.

In some embodiments the nucleic acid is operably linked to a transcriptional regulatory sequence functional in the animal, such as a constitutive or inducible promoter. In one embodiment the constitutive promoter is the short alpha tubulin promoter.

The bacterial organomercurial lyase and/or the bacterial mercuric reductase may be from one or more Aeropyrum, Caldivirga, Metallosphaera, Pyrobaculum, Sulfolobus, Thermoproteus, Ferroplasma, Haloarcula, Halorubrum, Picrophilus, Thermoplasma, Hydrogenivirga, Hydrogenobacter, Hydrogenobaculum, Acidimicrobium, Acidothermus, Aeromicrobium, Arthrobacter, Brevibacterium, Cellulomonas, Corynebacterium, Gordonia, Kytococcus, Micrococcus, Micromonospora, Mycobacterium, Nocardioides, Rubrobacter, Stackebrandtia, Streptomyces, Chryseobacterium, Leeuwenhoekiella, Rhodothermus, Sphingobacterium, Zunongwangia, Meiothermus, Thermus, Thermomicrobium, Aerococcus, Alicyclobacillus, Anoxybacillus, Bacillus, Clostridium, Enterococcus, Exiguobacterium, Geobacillus, Granulicatella, Bacillus, Staphylococcus, Streptococcus, Veillonella, Acholeplasma, Leptospirillum, Aurantimonas, Hyphomonas, Labrenzia, Maricaulis, Maritimibacter, Methylobacterium, Nisaea, Oceanibulbus, Oceanicola, Ochrobactrum, Octadecabacter, Oligotropha, Parvularcula, Roseovarius, Sphingopyxis, Sulfitobacter, Xanthobacter, Acidovorax, Alcaligenes, Burkholderia, Comamonas, Cupriavidus, Delftia, Gallionella, Janthinobacterium, Nitrosomonas, Polaromonas, Ralstonia, Thiobacillus, Thiomonas, Geobacter, Acidithiobacillus, Acinetobacter, Aeromonas, Alteromonas, Congregibacter, Enhydrobacter, Escherichia, Gamma proteobacterium, Haemophilus, Halothiobacillus, Idiomarina, Kangiella, Klebsiella, Marinobacter, Methylococcus, Methylophaga, Morganella, Nitrosococcus, Pantoea, Proteus, Pseudoalteromonas, Pseudomonas, Salmonella, Serratia, Shewanella, Shigella, Stenotrophomonas, Vibrio, Xanthomonas, Yersinia, Methylacidiphilum, or preferably Escherichia.

The heterologous nucleic acid sequence is the mer operon of a portion thereof, for example encoding the bacterial organomercurial resistance proteins organomercurial lyase and mercuric reductase.

In one embodiment the bacterial organomercurial lyase is Mer B, for example Mer B encoded by SEQ ID NO: 7 or a sequence at least 80%, 85%, 90%, 95%, 97%, or 99% identical to SEQ ID NO: 7.

In one embodiment the bacterial organomercurial lyase is Mer B, for example Mer B encoded by SEQ ID NO: 1 or a sequence at least 80%, 85%, 90%, 95%, 97%, or 99% identical to SEQ ID NO: 1.

In one embodiment the bacterial organomercurial lyase is Mer A, for example Mer A encoded by SEQ ID NO: 8 or a sequence at least 80%, 85%, 90%, 95%, 97%, or 99% identical to SEQ ID NO: 8.

In one embodiment the bacterial organomercurial lyase is Mer A, for example Mer A encoded by SEQ ID NO: 2 or a sequence at least 80%, 85%, 90%, 95%, 97%, or 99% identical to SEQ ID NO: 2.

The transgenic animal may be an insect, mammal, herbivore, fish, crustacean, mollusc, or sea sponge.

The insect is selected from the genus Hermetia, or Drosophila, for example Hermetia illucens, or Drosophila melanogaster.

The mammal may be a bovine, ovine, porcine, caprine, cervid, lagomorph, or camelid.

The fish may be a salmon, tuna, cod, trout, halibut, barramundi, kingfish, carp, tilapia, or catfish.

The crustacean may be a prawn, shrimp, lobster, or crayfish.

The mollusc may be a snail or slug (terrestrial or aquatic), mussel, oyster, scallop, limpet, abalone, squid, octopus, cuttlefish, cockle or clam.

In a second aspect there is provided a cell, tissue, or extract of the transgenic animal of the first aspect.

In a third aspect there is provided a method for bioremediation of material contaminated with mercury comprising:
(a) providing the material to a transgenic animal of the first aspect; and
(b) allowing the animal to feed on the material.

In a fourth aspect there is provided a method for preventing bioaccumulation of an organomercurial compound in an aquatic food chain comprising introducing the transgenic fish, crustacean, mollusc or sea sponge of the first aspect into the food chain.

### Definitions

Throughout this specification, unless the context clearly requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, the term ‘consisting essentially of’ means the inclusion of the stated element(s), integer(s) or step(s), but other element(s), integer(s) or step(s) that do not materially after or contribute to the working of the invention may also be included.

Throughout this specification, the term ‘consisting of’ means consisting only of.

Any discussion of documents, acts, materials, devices, articles or the like which has been included in the present specification is solely for the purpose of providing a context for the present technology. It is not to be taken as an admission that any or all of these matters form part of the prior art base or were common general knowledge in the field relevant to the present technology as it existed before the priority date of each claim of this specification.

Unless the context requires otherwise or specifically stated to the contrary, integers, steps, or elements of the technology recited herein as singular integers, steps or elements clearly encompass both singular and plural forms of the recited integers, steps, or elements.

In the context of the present specification the terms ‘a’ and ‘an’ are used to refer to one or more than one (ie, at least one) of the grammatical object of the article. By way of example, reference to ‘an element’ means one element, or more than one element.

In the context of the present specification the term ‘about’ means that reference to a figure or value is not to be taken as an absolute figure or value, but includes margins of variation above or below the figure or value in line with what a skilled person would understand according to the art, including within typical margins of error or instrument limitation. In other words, use of the term ‘about’ is understood to refer to a range or approximation that a person or skilled in the art would consider to be equivalent to a recited value in the context of achieving the same function or result.

A ‘promoter’ is defined as an array of nucleic acid control sequences that direct transcription of an operably linked nucleic acid. Promoters include necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription.

The term ‘operably linked’ refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

Those skilled in the art will appreciate that the technology described herein is susceptible to variations and modifications other than those specifically described. It is to be understood that the technology includes all such variations and modifications. For the avoidance of doubt, the technology also includes all of the steps, features, and compounds referred to or indicated in this specification, individually or collectively, and any and all combinations of any two or more of said steps, features and compounds.

In order that the present technology may be more clearly understood, preferred embodiments will be described with reference to the following drawings and examples.

### BRIEF DESCRIPTION OF THE DRAWINGS

FIG. 1: In vitro organomercurial lyase (MerB) methylmercury protonolysis in fly lysates. Fly lysate and controls were incubated with 1 μM methylmercury chloride in 50 mM sodium phosphate buffer pH 7.4 and 1 mM s-mercaptoethanol. The protonolysis product, inorganic mercury (Hg²⁺), was measured by cold-vapour atomic absorption spectroscopy. n=3 biologically independent replicates. NFC=No-Fly control, WT=wild-type D. melanogaster, Dme/Sa-MerB=D. melanogaster engineered with MerB from S. aureus, Dme/Ec-MerB=D. melanogaster engineered with MerB from E. coli. The statistical analysis was conducted using one-way ANOVA using Dunnett's method compared to WT, where ns=not significant.

FIG. 2: In vivo methylmercury detoxification in flies expressing organomercurial lyase and mercuric reductase. Dme/Ec-MerA+B and wild-type larvae were reared in cornmeal diet spiked with 200 parts per billion methylmercury chloride. Inorganic mercury (Hg²⁺) and total mercury (Hg²⁺ and organomercury) were measured by cold-vapour atomic absorption spectroscopy in 10 adult flies after eclosion or in 10 adult flies that were transferred to cornmeal diet without methylmercury for 48 h. n=2 for biologically independent replicates for Dme/Ec-MerA+B, n=3 for WT. WT=wild-type D. melanogaster, Dme/Ec-MerA+B=D. melanogaster engineered with MerA and MerB from Eschericia col. The statistical analysis was conducted using one-way ANOVA using Dunnett's method compared to WT, where ns=not significant.

FIG. 3: In vivo methylmercury detoxification into volatile Hg⁰ in flies expressing organomercurial lyase and mercuric reductase. Dme/Ec-MerA+B larvae and controls were reared in cornmeal diet spiked with 1000 ng mercury as methylmercury chloride. Hg⁰ volatilised in the sample headspace was amalgamated onto gold traps and measured by cold-vapour atomic absorption spectroscopy. n=2 for biologically independent replicates for NFC, WT, and Dme/Ec-MerB only; n=3 for Dme/Ec-MerA+B. NFC=No-Fly control, WT=wild-type D. melanogaster, Dme/Ec-MerB only=D. melanogaster engineered with MerB from Escherichia coli, Dme/Ec-MerA+B=D. melanogaster engineered with MerA and MerB from Escherichia coli. The statistical analysis was conducted using one-way ANOVA using Dunnett's method compared to WT, where ns=not significant.

### DESCRIPTION OF EMBODIMENTS

The technology described herein generally relates to transgenic animals for example insects, expressing bacterial MerA and MerB enzymes.

Microorganisms such as bacteria are involved in the global mercury cycle by reducing chemical forms of mercury (Hg²⁺, MeHg⁺) to the metallic form Hg(0). Reduced mercury (Hg⁰) is less soluble in aqueous systems and therefore less bioavailable. Metallic mercury is the less toxic form of all mercury species.

Reduction of mercury (II) forms to elemental mercury is widely distributed in Gram-positive and Gram-negative bacteria. Genes responsible for metal uptake and reduction are organized in operons present in plasmids and transposons. The merRTPABD cluster is a typical mer operon in Gram-negative bacteria, which confers resistance to mercury compounds. Mercury induces the expression of structural genes merTPABD. The expression of the mer genes is regulated by a transcriptional regulator encoded by the merR gene. MerR is a transcriptional regulator of the mer operon which acts as a repressor or activator in the absence or presence of mercury, respectively. MerD is a protein that is synthesized by the cell when the mercury has been completely removed from the cytoplasm and acts as a distal regulator. MerP is a periplasmic protein that captures extracellular mercury and transfer it to the MerT membrane protein, which delivers Hg(Ill) to the cytosolic protein MerA (mercuric reductase) that enzymatically reduces the ionic mercury to the metallic state. MerB is an organomercurial lyase that catalyses the protonolytic cleavage of carbon-mercury bonds in organomercurial compounds releasing Hg (II) for the reduction by MerA.

MerB has a wide substrate specificity and catalyzes the protonolysis of the C—Hg bond in a wide range of organomercurial salts (primary, secondary, tertiary, alkyl, vinyl, allyl, and aryl) to the hydrocarbon and mercuric ion. Accordingly, the transgenic animals described herein are useful for conversion of a wide range of organomercurial contaminants, such as methylmercury, dimethylmercury, and phenylmercuric acetate, into Hg(0).

Animals including insects such as Drosophila melanogaster can be genetically engineered to express mercuric reductase (MerA) and organomercurial lyase (MerB) from a bacterial species such as E. coli. The transgenic animals can then be used to remove mercury from biomass. Methylmercury is first protonolysed by MerB to inorganic Hg²⁺. MerB then passes Hg²⁺ to MerA. MerA accepts two electrons from NADPH to reduce Hg²⁺ to Hg(0). Each step results in a substantial reduction in toxicity and Hg(0) is a volatile form of mercury that can evaporate out of biomass.

Animals engineered to convert mercury compounds such as methylmercury and or Hg²⁺ to volatile Hg(0) can disrupt biomagnification of methylmercury and other mercury compounds, clean up organic waste streams with high mercury content, and bioremediate contaminated environments. In some embodiments, these animals may be insects capable of processing organic waste, such as black solider flies (Hermetia illucens), that are fed municipal biosolids or fisheries waste and producing fertilizers, animal feed, or other products with minimal mercury content. Other embodiments may be herbivores, such as goats (Capra hircus) that consume plants grown in soils contaminated with mercury thereby helping to remove mercury from the environment and producing meat or animal products that are safe to consume. Additional embodiments may be fish or aquatic invertebrates which disrupt mercury bioaccumulation. Some embodiments may involve allowing the animals to volatilize the Hg(0) into the atmosphere where it poses far less of a health risk while others may involve enclosures where volatilized Hg(0) is captured and stored.

The transgenic animals when released into environment would release volatile Hg(0) back into the biosphere but it would be removing mercury directly from the food chain where it is causing quantifiable harm. It would also dilute Hg before it is re-oxidised into Hg(II) and re-methylated to methylmercury before re-entering the food chain again.

Alternatively, in contained facilities, such as fish farms and insect waste processing facilities, the atmospheric mercury produced by the transgenic animals can be trapped and then disposed as hazardous waste thereby removing it from the biosphere. For example, in insect waste processing the spent uncontaminated organic waste can be used as a soil amendment and the uncontaminated pupae can be used as animal feed.

### Heterologous Nucleic Acids

The present invention provides a transgenic animal comprising heterologous nucleic acid encoding at least two bacterial mercury resistance proteins. As a result of expression of organomercurial lyase and/or mercuric reductase proteins the animal can reduce an inorganic mercury or an organomercurial compound to elemental mercury (Hg(0)).

MerA and MerB can be encoded by one or more heterologous nucleic acids. For example the heterologous nucleic acid may be all or a part of the Mer operon operably linked to transcriptional and translational control sequences which are functional in the animal.

Preferably the nucleic acid includes coding sequences for bacterial merA and merB, alternatively there are two heterologous nucleic acids one encoding the merA and the other encoding the merB. In the context of this disclosure, merA refers to mercuric reductase and refers to any enzyme comprised by the enzyme classification (EC:1.16.1.1). Similarly merB refers to any organomercurial lyase or alkylmercury lyase comprised by the enzyme classification (EC:4.99.1.2).

The merA and merB coding sequences are derived from any bacterial species. For example merA and/or merB sequences used in the transgenic animal can be derived from bacteria of the genera Aeropyrum, Caldivirga, Metallosphaera, Pyrobaculum, Sulfolobus, Thermoproteus, Ferroplasma, Haloarcula, Halorubrum, Picrophilus, Thermoplasma, Hydrogenivirga, Hydrogenobacter, Hydrogenobaculum, Acidimicrobium, Acidothermus, Aeromicrobium, Arthrobacter, Brevibacterium, Cellulomonas, Corynebacterium, Gordonia, Kytococcus, Micrococcus, Micromonospora, Mycobacterium, Nocardioides, Rubrobacter, Stackebrandtia, Streptomyces, Chryseobacterium, Leeuwenhoekiella, Rhodothermus, Sphingobacterium, Zunongwangia, Meiothermus, Thermus, Thermomicrobium, Aerococcus, Alicyclobacillus, Anoxybacillus, Bacillus, Clostridium, Enterococcus, Exiguobacterium, Geobacillus, Granulicatella, Bacillus, Staphylococcus, Streptococcus, Veillonella, Acholeplasma, Leptospirillum, Aurantimonas, Hyphomonas, Labrenzia, Maricaulis, Maritimibacter, Methylobacterium, Nisaea, Oceanibulbus, Oceanicola, Ochrobactrum, Octadecabacter, Oligotropha, Parvularcula, Roseovarius, Sphingopyxis, Sulfitobacter, Xanthobacter, Acidovorax, Alcaligenes, Burkholderia, Comamonas, Cupriavidus, Delftia, Gallionella, Janthinobacterium, Nitrosomonas, Polaromonas, Ralstonia, Thiobacillus, Thiomonas, Geobacter, Acidithiobacillus, Acinetobacter, Aeromonas, Alteromonas, Congregibacter, Enhydrobacter, Escherichia, Gamma proteobacterium, Haemophilus, Halothiobacillus, Idiomarina, Kangiella, Klebsiella, Marinobacter, Methylococcus, Methylophaga, Morganella, Nitrosococcus, Pantoea, Proteus, Pseudoalteromonas, Pseudomonas, Salmonella, Serratia, Shewanella, Shigella, Stenotrophomonas, Vibrio, Xanthomonas, Yersinia, Methylacidiphilum, or preferably Escherichia. In one embodiment the transgenic animals utilise merA and merB from E. coli. For example the merB may be encoded by SEQ ID NO: 1 or a sequence at least 80%, at least 85%, at least 90%, at least 95%, at least 97%, or at least 99% identical to SEQ ID NO: 1. In an embodiment merB may be encoded by SEQ ID NO: 7 or a sequence at least 80%, at least 85%, at least 90%, at least 95%, at least 97%, or at least 99% identical to SEQ ID NO: 7. The merA may be encoded by SEQ ID NO: 2, or a sequence at least 80%, at least 85%, at least 90%, at least 95%, at least 97%, or at least 99% identical to SEQ ID NO: 2. In an embodiment the merA may be encoded by SEQ ID NO: 8, or a sequence at least 80%, at least 85%, at least 90%, at least 95%, at least 97%, or at least 99% identical to SEQ ID NO: 8.

The merA and or merB may contain various sequence changes from the wild-type or naturally occurring merA or merB. In this context, the term “% identity” refers to the level of nucleic acid or amino acid sequence identity between the modified mer protein and the wild-type mer protein. For example, modified merA or merB may have 80%, 81%, 82%, 83%, 84%, 85%, 86%, 87%, 88%, 89%, 90%, 91%, 92%, 93%, 94%, 95%, 96%, 97%, 98% or 99% identity to their wild-type or codon optimised counterparts, for example SEQ ID NO: 1, SEQ ID NO: 2, SEQ ID NO: 7 or SEQ ID NO: 8. In some embodiments the modifications do not alter the enzymatic activity or specificity of the mercuric reductase or organomercurial lyase.

In some embodiments, it may be desirable to modify the mercuric reductase, organomercurial lyase or both. One of skill will recognize many ways of generating alterations in a given nucleic acid construct. Such well-known methods include site-directed mutagenesis, gene editing (e.g. CRISPR), PCR amplification using degenerate oligonucleotides, exposure of cells containing the nucleic acid to mutagenic agents or radiation, chemical synthesis of a desired oligonucleotide (e.g., in conjunction with ligation and/or cloning to generate a nucleic acid encoding a modified enzyme).

In some embodiments the nucleic acids encoding the mercuric reductase, organomercurial lyase or both may be conservatively modified. With respect to nucleic acid sequences, conservatively modified refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are ‘silent variations’ which are one species of conservatively modified variations. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a ‘conservatively modification’ where the alteration results in the substitution of an amino acid with a chemically similar amino acid. Conservative substitution tables providing functionally similar amino acids are well known in the art.

The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (1), Leucine (L), Methionine (M), Valine (V); and
6) Phenylalanine (F), Tyrosine (Y), Tryptophan (W).

A skilled person will recognise that other modifications can be made to the mercuric reductase and/or organomercurial lyase polypeptides or nucleic acids without diminishing their biological activity. Some modifications may be made to facilitate the cloning, expression, and the like. Such modifications are well known to those of skill in the art and include, for example, a methionine added at the amino terminus to provide an initiation site, or additional amino acids that form an epitope tag (e.g., poly His) placed on either terminus to facilitate purification or identification.

In some embodiments the merA and/or merB sequences are codon optimised for the target animal in which they are expressed. As used herein, the term “codon-optimised” means a nucleic acid protein coding sequence has been adapted for expression in a target animal (for example an insect or mammal) by substitution of one or more, preferably a significant number of codons with codons that are more frequently used in the target animal.

In some embodiments the merB is codon optimised for D. melanogaster and is encoded by SEQ ID NO: 7, or a sequence at least 80%, at least 85%, at least 90%, at least 95%, at least 97%, or at least 99% identical to SEQ ID NO: 7. In some embodiments the merA is codon optimised for D. melanogaster and is encoded by SEQ ID NO: 8, or a sequence at least 80%, at least 85%, at least 90%, at least 95%, at least 97%, or at least 99% identical to SEQ ID NO: 8.

In another preferred embodiment, the percentage of optimised codons is, in increasing order of preference, at least 10%, 20%, 30%, 40%, 50%, 60%, 70%, 80%, 90%, 95%, 98%, 99% optimised. In a particular embodiment each and every codon position is optimised for the target animal. The codon-optimised sequences can be synthesised chemically using any known technique in the art.

Manipulating the properties of the mercuric reductase and/or organomercurial lyase described herein can be achieved using any protein engineering methods known in the art. In order to express the mercuric reductase an organomercurial lyase in a target animal such as an insect, a nucleic acid encoding the both the mercuric reductase and organomercurial lyase separately or transcriptionally/translationally fused or separate nucleic acids each encoding the mercuric reductase or the organomercurial lyase is incorporated into an expression cassette or expression vector. A typical expression cassette, which may be part of a larger nucleic acid construct such as an expression vector, contains a promoter operably linked to a nucleic acid encoding the mercuric reductase and/or organomercurial lyase and optionally other sequences such as a transcription terminator.

The promoter can be constitutive or inducible. Inducible promoters can be advantageous because the animals can be maintained in conditions without inorganic mercury or organomercurial contamination before expression of the mercuric reductase and organomercurial lyase is induced when the animal is exposed to a feed or environment with inorganic mercury or organomercurial contamination.

Examples of suitable constitutive promoters for use in insects are actin 5C promoter, or the short tubulin alpha promoter. In one embodiment the short tubulin alpha promoter is used.

The transgenic insects exemplified herein utilise the shortened alpha tubulin promoter to drive midlevel expression across all tissues. This promoter avoids potential toxicity arising from overexpression. As the shortened alpha tubulin promoter is effective it is apparent that a strong ubiquitous promoter that drives the overexpression of enzymes in all tissues can also be used.

Suitable inducible promoters include the mer operator/promoter region. The metal-free, apo MerR binds to the mer operator/promoter region as a repressor to block transcription initiation, but is converted into an activator upon Hg²⁺-binding. In this embodiment the transgenic animal may express active MerR.

### Animals

One advantage of the present invention is that the bacterial mercuric reductase and organomercurial lyase can be expressed in insect species that can be easily cultivated on food or agricultural waste contaminated by inorganic mercury or an organomercurial compound. Accordingly, any insect used for “bioconversion” of waste into insect biomass can be used to express the mercuric reductase and organomercurial lyase. A suitable insect is the black soldier fly (Hermetia illucens) being the a commonly used species for bioconversion of organic waste. However, it is envisaged that any insect species amenable to genetic modification can be used to express merA and merB. Considering the diversity of materials potentially contaminated by inorganic mercury or an organomercurial compounds it is envisaged that material-to-insect pairings to maximize both bioconversion and insect biomass will be made by the skilled person.

The insects may be cockroaches, flies, beetles, worms, larval stages of other flying insects such as meal worms, caterpillars, etc. The most desirable insects to produce mercuric reductase and organomercurial lyase in terms of composition, size, reproduction, palatability, and lack of known toxins are typically species found within the orders Blattodea (cockroaches), Orthoptera (grasshoppers, locusts, katydids, crickets), Diptera (flies), and Lepidoptera (moths and butterflies).

It is envisaged that any dipteran insect may be used to express the mercuric reductase and organomercurial lyase. Suitable dipteran insects include soldier flies, robber flies, bee flies, hover flies, fruit flies, dragon flies, vinegar flies, and blowflies.

In one embodiment, the insect is a fruit fly (Drosophila sp.), black soldier fly (Hermetia sp, for example Hermetia illucens), or house fly. Preferably the insect is a black soldier fly.

In other embodiments, the insect may be a mealworm, for example of the genus Tenebrio. In one embodiment the mealworm is Tenebrio molitor.

The mercuric reductase and organomercurial lyase can be expressed in one or any combination of the eggs, larvae, pupae, and adults.

The transformation of insects with heterologous nucleic acids is known in the art and is a process in which exogenous DNA sequences are introduced into the insect germ line. Numerous methods for transforming insects and nucleic acid vectors that can be used for insect transformation are known in the art and can be used in the present invention.

Other animals can also be transformed with bacterial mercuric reductase and organomercurial lyase. These include mammals, fish, crustaceans, and molluscs.

It is envisaged that mammalian species common in agriculture can be transformed. For example the mammal may be a rodent, bovine such as cattle or buffalo, ovine (sheep), porcine (pigs, wild boar), caprine (goats), cervid (deer), lagomorph (hares and rabbits), or camelids (camels, llamas, alpacas).

The expression of mercuric reductase and organomercurial lyase in mammals provides the mammals with the ability to convert organomercurial contaminants in feed or the environment into gaseous monatomic Hg(0) vapor which is volatilised to the atmosphere. Accordingly, meat from the mammals may have little to no detectable mercury and may be safe for human consumption even when the animals are raised on contaminated land or provided with feed having organomercurial or Hg(II) contamination. Species such as fish, crustaceans, and molluscs may also be genetically modified to express bacterial mercuric reductase and organomercurial lyase to provide these animals with the ability to convert organomercurial or inorganic mercury contaminants in feed or the environment into gaseous monatomic Hg(0) vapor.

Suitable fish species include any species raised in aquaculture. For example the fish may be salmon, tuna, cod, trout, halibut, barramundi, kingfish, carp, tilapia, or catfish.

Similarly, crustaceans such prawns, shrimps, lobster, or crayfish may also be modified to express bacterial mercuric reductase and organomercurial lyase.

Terrestrial and aquatic molluscs may also be modified to express bacterial mercuric reductase and organomercurial lyase. Suitable molluscs include snails and slugs (both terrestrial and aquatic), mussels, oysters, scallops, limpets, abalone, squid, octopus, cuttlefish, cockles or clams.

In one embodiment the invention provides a cell, tissue, or extract of the transgenic animal.

### Methods

The invention provides various methods of using the transgenic animals to remove inorganic mercury or organomercurial contaminants or to utilise materials contaminated by inorganic mercury or organomercuric compounds.

In one aspect there is provided a method for bioremediation of material contaminated with inorganic mercury or an organomercurial compound. The method requires providing the contaminated material to a transgenic animal described herein and allowing the animal to feed on the material.

For example, the material may be a contaminated feed, or the material may be a pasture grown on contaminated land and which has accumulated mercury from the environment. In other embodiment the material may be a contaminated organic waste and the animal may be a transgenic insect that feeds on the waste.

In another aspect there is provided a method for preventing bioaccumulation of inorganic mercury or an organomercurial compound in a food chain, such as an aquatic food chain. In this aspect the method involves introducing the transgenic fish, crustacean or mollusc into the food chain. Once part of the food chain the transgenic animal, will be virtue of expressing the bacterial mercuric reductase and organomercurial lyase to convert inorganic mercury or organomercurial compounds into gaseous monatomic Hg(0) vapor.

In an aquatic food chain this decontamination will occur whether the inorganic mercury or organomercurial contamination is present in the food ingested by the transgenic animal, in the water in which the animal lives, or both.

In some embodiments the extract of the transgenic animal, in particular extracts from insects such as those grown on waste products can be used in the field of waste-water treatment. For example, the preparations will contain active bacterial mercuric reductase and organomercurial lyase and can be used convert organomercurial compounds in waste water into gaseous monatomic Hg(0) vapor.

In some embodiments the extracts can reduce organomercurial contamination by about 5%, 10%, 15%, 20%, 25%, 30%, 35%, 40%, 45%, 50%, 55%, 56%, 60%, 65%, 70%, 75%, 80%, 85% or at least about 90%, for example compared to a control extract that lacks mercuric reductase and organomercurial lyase.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### EXAMPLES

### Example 1: Methods and Results

Two MerA and two MerB variants were synthesised from Escherichia coli and Staphylococcus aureus. The sequences were codon optimized (SEQ ID NO: 7 and SEQ ID NO: 8) for D. melanogaster, and expression is controlled by a short variant of the D. melanogaster α-tubulin promoter. These plasmids are designated pKT111.EcMerA (SEQ ID NO: 3), pKT111.EcMerB (SEQ ID NO: 4), for the E. coli MerA and MerB variants, respectively. pKT111.SaMerA (SEQ ID NO: 5), pKT111.SaMerB (SEQ ID NO: 6) were designated for the S. aureus MerA and MerB variants, respectively. pKT111.EcMerA (SEQ ID NO: 3), includes SEQ ID NO: 8 and pKT111.EcMerB (SEQ ID NO: 4), includes SEQ ID NO: 7. Transgenic D. melanogaster were generated via standard embryo microinjection and PhiC31 mediated integration methods.

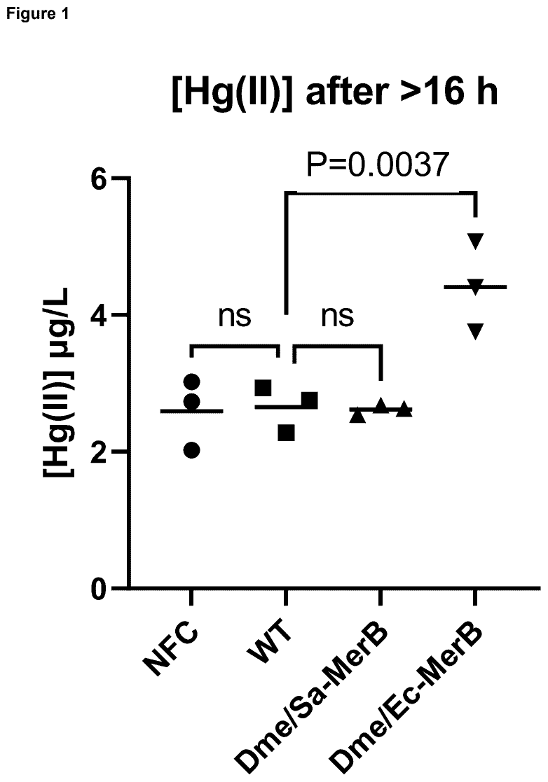

The function of MerB activity was evaluated in vitro by incubating fresh fly lysates in buffer containing methylmercury, and measured the breakdown product Hg²⁺ using cold-vapour atomic absorption spectroscopy. No activity was detected from wild-type or S. aureus MerB fly lysates, however, E. coli MerB fly lysates showed detectable levels of activity (FIG. 1). In vivo assays were performed to test E. coli MerA and MerB when combined in the same organism.

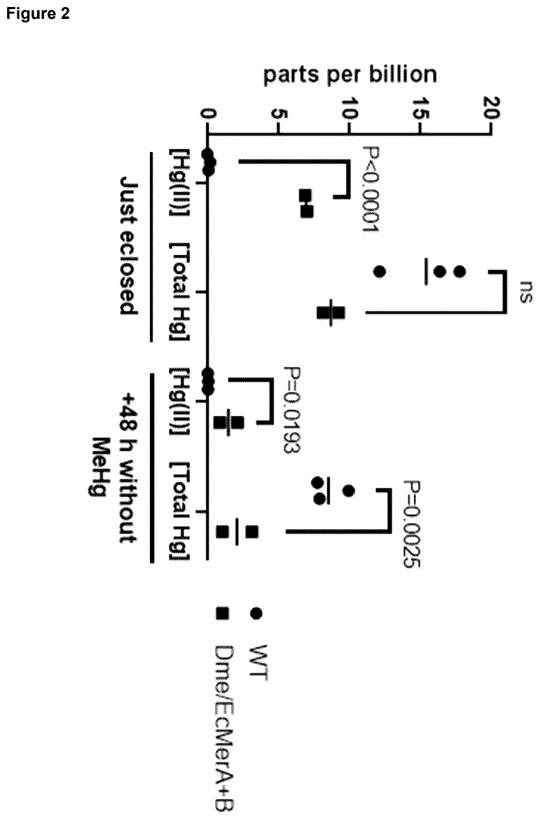

Flies expressing E. coli MerA+MerB or wild-type controls were reared in media containing methylmercury and assayed for Hg²⁺ and total mercury (inorganic Hg²⁺ and organomercury) content shortly after emerging from pupae (enclosed) or after being transferred to media without methylmercury for 48 hrs (FIG. 2). Only transgenic flies contained Hg²⁺ which demonstrates that MerB is functioning in vivo. Although there is no significant difference in the amount of total mercury shortly after adults emerge, after two days, the transgenic flies have eliminated almost all of the mercury which supports that MerA is functioning.

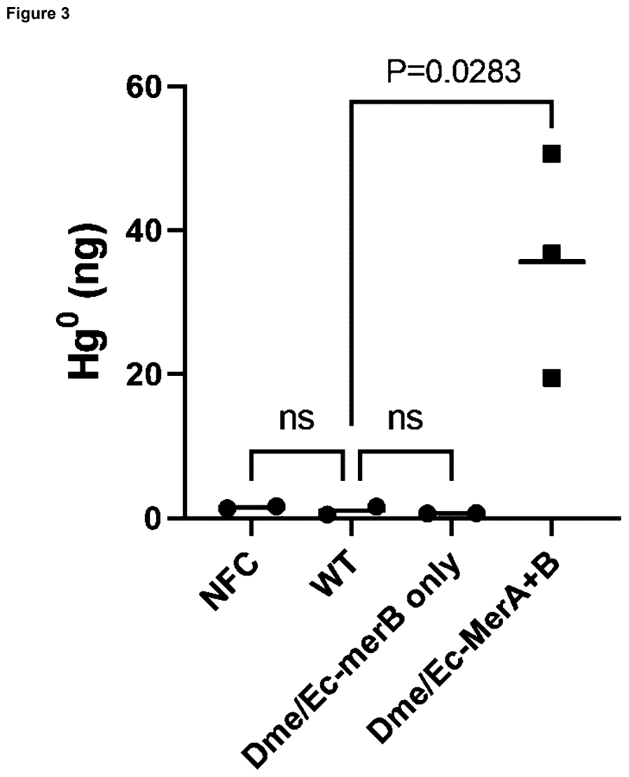

Flies expressing E. coli MerA+MerB or controls were reared in media containing methylmercury and assayed for Hg⁰ volatilisation into the sample vial headspace for 48 hrs (FIG. 3). Transgenic flies with E. coli MerA+MerB volatilised statistically significantly more Hg⁰ compared to controls, which demonstrates that both MerB and MerA are functional in vivo.

1-25. (canceled)

26. A transgenic animal comprising heterologous nucleic acids encoding a bacterial organomercurial lyase and/or a bacterial mercuric reductase, or a cell, tissue, or extract of the transgenic animal, wherein the transgenic animal expresses the bacterial organomercurial lyase and/or a bacterial mercuric reductase to reduce the toxicity of a mercury compound.

27. The transgenic animal of claim 26, wherein the heterologous nucleic acid encodes the bacterial organomercurial lyase and the mercury compound is an organomercurial compound.

28. The transgenic animal of claim 26, wherein the heterologous nucleic acid encodes the bacterial mercuric reductase and the mercury compound is an inorganic mercury compound.

29. The transgenic animal of claim 26, wherein the heterologous nucleic acid encodes the bacterial organomercurial lyase and the bacterial mercuric reductase and the mercury compound is an organomercurial compound and/or an inorganic mercury compound.

30. The transgenic animal of claim 26, wherein the nucleic acid is operably linked to a transcriptional regulatory sequence functional in the transgenic animal.

31. The transgenic animal of claim 26, wherein the bacterial organomercurial lyase and/or the bacterial mercuric reductase is from one or more of Aeropyrum, Caldivirga, Metallosphaera, Pyrobaculum, Sulfolobus, Thermoproteus, Ferroplasma, Haloarcula, Halorubrum, Picrophilus, Thermoplasma, Hydrogenivirga, Hydrogenobacter, Hydrogenobaculum, Acidimicrobium, Acidothermus, Aeromicrobium, Arthrobacter, Brevibacterium, Cellulomonas, Corynebacterium, Gordonia, Kytococcus, Micrococcus, Micromonospora, Mycobacterium, Nocardioides, Rubrobacter, Stackebrandtia, Streptomyces, Chryseobacterium, Leeuwenhoekiella, Rhodothermus, Sphingobacterium, Zunongwangia, Meiothermus, Thermus, Thermomicrobium, Aerococcus, Alicyclobacillus, Anoxybacillus, Bacillus, Clostridium, Escherichia, Enterococcus, Exiguobacteium, Geobacillus, Granulicatella, Bacillus, Staphylococcus, Streptococcus, Veillonella, Acholeplasma, Leptospirillum, Aurantimonas, Hyphomonas, Labrenzia, Maricaulis, Maritimibacter, Methylobacterium, Nisaea, Oceanibulbus, Oceanicola, Ochrobactrum, Octadecabacter, Oligotropha, Parvularcula, Roseovarius, Sphingopyxis, Sulfitobacter, Xanthobacter, Acidovorax, Alcaligenes, Burkholderia, Comamonas, Cupriavidus, Delftia, Gallionella, Janthinobacterium, Nitrosomonas, Polaromonas, Ralstonia, Thiobacillus, Thiomonas, Geobacter, Acidithiobacillus, Acinetobacter, Aeromonas, Alteromonas, Congregibacter, Enhydrobacter, Escherichia, Gamma proteobacterium, Haemophilus, Halothiobacillus, Idiomarina, Kangiella, Klebsiella, Marinobacter, Methylococcus, Methylophaga, Morganella, Nitrosococcus, Pantoea, Proteus, Pseudoalteromonas, Pseudomonas, Salmonella, Serraia, Shewanella, Shigella, Stenotrophomonas, Vibrio, Xanthomonas, Yersinia, Methylacidiphilum, preferably Escherichia.

32. The transgenic animal of claim 26, wherein the heterologous nucleic acid sequence is a mer operon, or a portion thereof.

33. The transgenic animal of claim 26, wherein the bacterial organomercurial lyase is Mer B.

34. The transgenic animal of claim 33, wherein the Mer B is encoded by SEQ ID NO: 7 or SEQ ID NO: 1, or a sequence that is at least 80%, 85%, 90%, 95%, 97%, or 99% identical to SEQ ID NO: 7 or SEQ ID NO: 1.

35. The transgenic animal of claim 26, wherein the bacterial mercuric reductase is Mer A.

36. The transgenic animal of claim 35, wherein the Mer A is encoded by SEQ ID NO: 8 or SEQ ID NO: 2, or a sequence that is at least 80%, 85%, 90%, 95%, 97%, or 99% identical to SEQ ID NO: 8 or SEQ ID NO: 2.

37. The transgenic animal of claim 30, wherein the transcriptional regulatory sequence is a constitutive or inducible promoter.

38. The transgenic animal of claim 37, wherein the constitutive promoter is the short alpha tubulin promoter.

39. The transgenic animal of claim 26, wherein the transgenic animal is selected from an insect, mammal, fish, crustacean, mollusc, or sea sponge.

40. The transgenic animal of claim 39, wherein the insect is selected from the genus Hermetia or Drosophila.

41. The transgenic animal of claim 40, wherein the insect is Hermetia illucens or Drosophila melanogaster.

42. The transgenic animal of claim 39, wherein the mammal is selected from a bovine, ovine, porcine, caprine, cervid, lagomorph, or camelid, the fish is a salmon, tuna, cod, trout, halibut, barramundi, kingfish, carp, tilapia, or catfish, the crustacean is a prawn, shrimp, lobster, or crayfish, or the mollusc is a snail, mussel, oyster, scallop, limpet, abalone, squid, octopus, cuttlefish, cockle or clam.

43. A method for bioremediation of material contaminated with an organomercurial compound comprising:
(a) providing the material to a transgenic animal of claim 26; and
(b) allowing the animal to feed on the material.

44. A method for preventing bioaccumulation of an organomercurial compound in an aquatic food chain comprising introducing the transgenic animal of claim 39 into the food chain.


SEQUENCE LISTING (8 sequences)

SEQ ID NO: 1 - DNA, 639 bp, synthetic construct
  source 1..639: mol_type = other DNA
atgaagctcgccccatatattttagaacttctcacttcggtcaatcgtaccaatggtactgcggatctcttggtcccgctactgcgggaactcgccaaggggcgtccggtttcacgaacgacacttgccgggattctcgactggcccgctgagcgagtggccgccgtactcgaacaggccaccagtaccgaatatgacaaagatgggaacatcatcggctacggcctcaccttgcgcgagacttcgtatgtctttgaaattgacgaccgccgtctgtatgcctggtgcgcgctggacaccttgatatttccggcgctgatcggccgtacagctcgcgtctcatcgcattgcgctgcaaccggagcaccggtttcactcacggtttcacccagcgagatacaggctgtcgaacctgccggcatggcggtgtccttggtattgccgcaggaagcagccgacgttcgtcagtccttctgttgccatgtacatttctttgcatctgtcccgacggcggaagactgggcctccaagcatcaaggattggaaggattggcgatcgtcagtgtccacgaggctttcggcttgggccaggagtttaatcgacatctgttgcagaccatgtcatctaggacaccgtga

SEQ ID NO: 2 - DNA, 1695 bp, synthetic construct
  source 1..1695: mol_type = other DNA
atgagcactctcaaaatcaccggcatgacttgcgactcgtgcgcagtgcatgtcaaggacgccctggagaaagtgcccggcgtgcaatcagcggatgtctcctacgccaagggcagcgccaagctcgccattgaggtcggcacgtcacccgacgcgctgacggccgctgtagctggactcggttatcgggccacgctggccgatgccccctcagtttcgacgccgggcggattgctcgacaagatgcgcgatctgctgggcagaaacgacaagacgggtagcagcggcgcattgcatatcgccgtcatcggcagcggcggggccgcgatggcagcggcgctgaaggccgtcgagcaaggcgcacgtgtcacgctgatcgagcgcggcaccatcggcggcacctgcgtcaatgtcggttgtgtgccgtccaagatcatgatccgcgccgcccatatcgcccatctgcgccgggaaagcccgttcgatggcggcatcgccgctaccacgccgaccatccagcgcacggcgctgctggcccagcagcaggcccgcgtcgatgaactgcgccacgccaagtacgaaggcatcttggagggcaatccggcgatcactgtgctgcacggctccgcccgctttaaggacaatcgcaacctgatcgtgcaactcaacgacggcggcgagcgcgtggtggcattcgaccgctgcctgatcgccaccggcgcgagcccggccgtgccgccgattcccggcctgaaagacactccgtactggacttccactgaagcgctggtcagcgagacgattcctaagcgcctggccgtgattggctcatcagtggtggcgctggagctggcgcaggcgttcgcccgactcggagcgaaggtgacgatcctggctcgcagcacgctgttcttccgcgaagacccagctataggcgaagccgtcacggccgcattccgcatggagggcatcgaggtgagggaacacacccaggccagccaggtcgcgtatatcaatggtgaaggggacggcgaattcgtgctcaccacggcgcacggcgaactgcgcgccgacaagctgctggtcgccaccggccgcgcgcccaacacacgcaagctggcactggatgcgacgggcgtcacgctcaccccgcaaggcgctatcgtcatcgaccccggcatgcgtacaagcgtggaacacatctacgccgcaggcgactgcaccgaccagccgcagttcgtctatgtggcggcagcggccggcactcgcgccgcgatcaacatgaccggcggtgacgcggccctgaacctgaccgcgatgccggccgtggtgttcaccgacccgcaagtggcgaccgtaggctacagcgaggcggaagcgcaccatgacggcatcaaaactgatagtcgcacgctaacgctggacaacgtgccgcgcgcgctcgccaacttcgacacgcgcggcttcatcaaactggtggttgaagaaggcagcggacgactgatcggcgtgcaggcagtggccccggaagcgggcgaactgatccagacggccgcactggcgattcgcaaccggatgacggtgcaggaactggccgaccagttgttcccctacctgacgatggtcgaagggttgaagctcgcggcgcagaccttcaacaaggatgtgaagcagctttcctgctgcgccgggtga

SEQ ID NO: 3 - DNA, 10419 bp, synthetic construct
  source 1..10419: mol_type = other DNA
aaaataatcaattaggggttccgcgcaacaatttccccgaaaaaagtgcccacctaaattgtaaagcgtttaatatttttgtttaaaattcgcgttaaattttttgttaaatcagctcatttttttaaccaataggccgaaaatcggcaaaaatcccttataaatcaaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacgtaccgggcccctagtatgtatgtaagttaataaaacccatttttgcggaaagtagataaaaaaaacattttttttttttactgcactggatatcattgaacttatctgatcagttttaaatttacttcgatccaagggtatttgatgtaccaggttctttcgattacctctcactcaaaatgacattccactcaaagtcagcgctgtttgcctccttctctgtccacagaaatatcgccgtctctttcgccgctgcgtccgctatctctttcgccaccgtttgtagcgttacgtagcgtcaatgtccgccttcagttgcattttgtcagcggtttcgtgacgaagctccaagcggtttacgccatcaattaaacacaaagtgctgtgccaaaactcctctcgcttcttatttttgtttgttttttgagtgattggggtggtgattggttttgggtgggtaagcaggggaaagtgtgaaaaatcccggcaatgggccaagaggatcaggagctattaattcgcggaggcagcaaacacccatctgccgagcatctgaacaatgtgagtagtacatgtgcatacatcttaagttcacttgatctataggaactgcgattgcaacatcaaattgtctgcggcgtgagaactgcgacccacaaaaatcccaaaccgcaattgcacaaacaaatagtgacacgaaacagattattctggtagctgttctcgctatataagacaatttttgagatcatatcatgatcaagacatctaaaggcattcattttcgactatattcttttttacaaaaaatataacaaccagatattttaagctgatcctagatgcacaaaaaataaataaaagtataaacctacttcgtaggatacttcggggtactttttgttcggggttagatgagcataacgcttgtagttgatatttgagatcccctatcattgcagggtgacagcggagcggcttcgcagagctgcattaaccagggcttcgggcaggccaaaaactacggcacgctccggccacccagtccgccggaggactccggttcagggagcggccaactagccgagaacctcacctatgcctggcacaatatggacatctttggggcggtcaatcagccgggctccggatggcggcagctggtcaaccggacacgcggactattctgcaacgagcgacacataccggcgcccaggaaacatttgctcaagaacggtgagtttctattcgcagtcggctgatctgtgtgaaatcttaataaagggtccaattaccaatttgaaactcagtttgcggcgtggcctatccgggcgaacttttggccgtgatgggcagttccggtgccggaaagacgaccctgctgaatgcccttgcctttcgatcgccgcagggcatccaagtatcgccatccgggatgcgactgctcaatggccaacctgtggacgccaaggagatgcaggccaggtgcgcctatgtccagcaggatgacctctttatcggctccctaacggccagggaacacctgattttccaagccatggtgcggatgccacgacatctgacctatcggcagcgagtggcccgcgtggatcaggtgatccaggagctttcgctcagcaaatgtcagcacacgatcatcggtgtgcccggcagggtgaaaggtctgtccggcggagaaaggaagcgtctggcattcgcctccgaggctctaaccgatccgccgcttctgatctgcgatgagcccacctccggactggactcctttaccgcccacagcgtcgtccaggtgctgaagaagctgtcgcagaagggcaagaccgtcatcctgaccattcatcagccgtcttccgagctgtttgagctctttgacaagatccttctgatggccgagggcagggtagctttcttgggcactcccagcgaagccgtcgacttcttttcctagtgagttcgatgtgtttattaagggtatctagtattacataacatctcaactcctatccagcgtgggtgcccagtgtcctaccaactacaatccggcggacttttacgtacaggtgttggccgttgtgcccggacgggagatcgagtcccgtgatcggatcgccaagatatgcgacaattttgccattagcaaagtagcccgggatatggagcagttgttggccaccaaaaatctggagaagccactggagcagccggagaatgggtacacctacaaggccacctggttcatgcagttccgggcggtcctgtggcgatcctggctgtcggtgctcaaggaaccactcctcgtaaaagtgcgacttattcagacaacggtgagtggttccagtggaaacaaatgatataacgcttacaattcttggaaacaaattcgctagattttagatagaattgcctgattccacacccttcttagtttttttcaatgagatgtatagtttatagttttgcagaagataaataaatttcatttaactcgcgaatattaatgagatgcgagtaacattttaatttgcagatggttgccatcttgattggcctcatctttttgggccaacaactcacgcaagtgggtgtgatgaatatcaacggagccatcttcctcttcctgaccaacatgacctttcaaaacgtctttgccacgataaatgtaagtcatgtttagaatacatttgcatttcaataatttactaactttctaatgaatcgattcgatttaggtgttcacctcagagctgccagtttttatgagggaggcccgaagtcgactttatcgctgtgacacatactttctgggcaaaacgattgccgaattgccgctttttctcacagtgccactggtcttcacggcgattgcctatccgatgatcggactgcgggccggagtgctgcacttcttcaactgcctggcgctggtcactctggtggccaatgtgtcaacgtccttcggatatctaatatcctgcgccagctcctcgacctcgatggcgctgtctgtgggtccgccggttatcataccattcctgctctttggcggcttcttcttgaactcgggctcggtgccagtatacctcaaatggttgtcgtacctctcatggttccgttacgccaacgagggtctgctgattaaccaatgggcggacgtggagccgggcgaaattagctgcacatcgtcgaacaccacgtgccccagttcgggcaaggtcatcctggagacgcttaacttctccgccgccgatctgccgctggactacgtgggtctggccattctcatcgtgagcttccgggtgctcgcatatctggctctaagacttcgggcccgacgcaaggagtagccgacatatatccgaaataactgcttgttttttttttttaccattattaccatcgtgtttactgtttattgccccctcaaaaagctaatgtaattatatttgtgccaataaaaacaagatatgacctatagaatacaagtatttccccttcgaacatccccacaagtagactttggatttgtcttctaaccaaaagacttacacacctgcataccttacatcaaaaactcgtttatcgctacataaaacaccgggatatattttttatatacatacttttcaaatcgcgcgccctcttcataattcacctccaccacaccacgtttcgtagttgctctttcgctgtctcccacccgctctccgcaacacattcaccttttgttcgacgaccttggagcgactgtcgttagttccgcgcgattcggttcgctcaaatggttccgagtggttcatttcgtctcaatagaaattagtaataaatatttgtatgtacaatttatttgctccaatatatttgtatatatttccctcacagctatatttattctaatttaatattatgactttttaaggtaattttttgtgacctgttcggagtgattagcgttacaatttgaactgaaagtgacatccagtgtttgttccttgtgtagatgcatctcaaaaaaatggtgggcataatagtgttgtttatatatatcaaaaataacaactataataataagaatacatttaatttagaaaatgcttggatttcactggaactaggctagcataacttcgtataatgtatgctatacgaagttatgctagcggatccgggaattgggaattcacgtaagtactgtctgcagcgtaagcttcgtacgtagcgaccgtctcaaagtactgcctttctgcgttggaaaacatcgcctttttcgtccaaaaggagtccccaggttcgatccgcatggcgttgtgcgtgcgtgcctttcttttcaaatgattacggctattaacttgggggcgttaagttggaaacacgtaaattgcagactgcgattagagtgaccatgagtaggagttcaaaatctcctgacatcattttcttaaaacctgctttgttttttacatttctatttaatataactcctatttgaataaaaaaacaaaacaagtttagatgttaagatattaactacatcctttgctccaaagggagaggggaagttatggagttaattaatttgctgttggaaatcaatatggagtcagaaatataatgatttactaaaccttattgaatcggtaacgatgcgaatttatattaaaatagcttttatgaaacattcaacaaaaatattattaatgttggcccactttagcaaccggttaggtctaccggttgggcaagcaaagattcacgccctggttcgagtcccaactagtcctgcaaaataccgcagcaagttttagagagaccaagtgccattacctctcccacttcagttatcggttatgcggcgtttaagtcgacagcttgccgtctctagctccggtgcctatataaagcagcccgctttccacatttcatattcgttttacgtttgtcaagcctcatagccggcagttcgaacgtatacgctctctgagtcagacctcgaaatcgtagctctacacaattctgtgaattttccttgtcgcgtgtgaaacacttccaaggcaatcttacaaaatgtcgaccctcaagatcactggtatgacctgcgatagctgtgcagtccacgtaaaggatgcgctcgaaaaggtcccgggtgtccaatccgctgacgtgtcctacgcaaaaggttccgctaagctggcaattgaagtaggaacgtcgcccgatgcactcactgccgccgtagcaggactgggatatagggcgacactcgccgacgcagcatcggtctcgactcctggaggtctgttggataaaatgcgggacctgttgggtcgtaacgacaaaactggaagcagtggagctctgcatatagcagttattggttcgggtggcgcagcaatggccgcggccctgaaggccgtggagcaaggcgcacgggtcactctgattgaacgaggtacgatcggtggaacttgcgtgaatgtaggttgcgtcccttccaaaataatgattcgtgcagcacatatagcgcatctccgtcgtgaatcgccctttgacggcggaattgctgccacgactcccaccatccagcgaacagcactgctcgcccagcaacaagcgagggtagatgagctcaggcacgcaaagtacgaaggcattctcgagggcaaccctgccattacagttttgcatggcagtgcacgcttcaaggacaaccgtaacctgattgtgcagctgaatgacggcggcgaacgagtcgtggccttcgacaggtgcctcatcgccaccggagcgtcccccgcagtgccgccaatacctggtttgaaagacaccccctattggacatccacagaagccttggttagtgaaacaattccgaagcgcctggcagtaattggtagctcggttgtagctctcgaattggcgcaggcgtttgcgcgactgggagcgaaggtaacaattttggcccgcagtaccttgttttttcgagaagacccagctattggcgaggcggtcacagctgccttccggatggagggtatcgaggttcgagagcacactcaggcatcccaggtagcctatatcaatggcgagggtgatggcgagttcgtcctgaccaccgctcatggcgagctccgggccgataagctgctggttgcgacaggccgagcgccaaatacccgcaagctggcattggatgcaacaggcgtaacgttgacgcctcaaggagcgatcgttatcgatccgggtatgcgcacatccgtcgagcacatttatgctgcaggcgattgtacggaccagcctcaattcgtctatgttgccgcggccgctggtacacgcgctgccataaacatgactggtggcgacgctgcgttgaatctcacagcgatgcccgccgtggtgttcacagacccccaggtcgctacagtcggttatagcgaagccgaagctcatcacgacggcatcaagaccgacagccgcacattgaccctcgataatgtccctagggcgctcgctaatttcgatacgcgtggattcattaaactcgtagtcgaagaaggaagcggtcgcttgatcggcgtgcaagcagttgctccagaagcgggtgagctcattcaaacggccgcactcgccattcggaatcgcatgacagtacaggagctggctgatcaactgtttccctatctcacgatggtagagggcctcaagctcgccgcacagacgttcaacaaagatgtgaagcaactcagttgctgtgccggctaagcccgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgcccaaactcatcaatgtatcttagcggctcgagggtacctctagagatccactagtgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacactagttctagccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttcctccgaaaagtgccacctaaattgtaagcgttaatatttttgttaaaattcgcgttaaattttttgttaaatcagctcatttttttaaccaataggccgaaatcggcaaaaatccccttataaatcaaaaagaatagaccgagatagggttgagtgttgttcccagttttggc

SEQ ID NO: 4 - DNA, 9363 bp, synthetic construct
  source 1..9363: mol_type = other DNA
aaaataatcaattaggggttccgcgcaacaatttccccgaaaaaagtgcccacctaaattgtaaagcgtttaatatttttgtttaaaattcgcgttaaattttttgttaaatcagctcatttttttaaccaataggccgaaaatcggcaaaaatcccttataaatcaaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacgtaccgggcccctagtatgtatgtaagttaataaaacccatttttgcggaaagtagataaaaaaaacattttttttttttactgcactggatatcattgaacttatctgatcagttttaaatttacttcgatccaagggtatttgatgtaccaggttctttcgattacctctcactcaaaatgacattccactcaaagtcagcgctgtttgcctccttctctgtccacagaaatatcgccgtctctttcgccgctgcgtccgctatctctttcgccaccgtttgtagcgttacgtagcgtcaatgtccgccttcagttgcattttgtcagcggtttcgtgacgaagctccaagcggtttacgccatcaattaaacacaaagtgctgtgccaaaactcctctcgcttcttatttttgtttgttttttgagtgattggggtggtgattggttttgggtgggtaagcaggggaaagtgtgaaaaatcccggcaatgggccaagaggatcaggagctattaattcgcggaggcagcaaacacccatctgccgagcatctgaacaatgtgagtagtacatgtgcatacatcttaagttcacttgatctataggaactgcgattgcaacatcaaattgtctgcggcgtgagaactgcgacccacaaaaatcccaaaccgcaattgcacaaacaaatagtgacacgaaacagattattctggtagctgttctcgctatataagacaatttttgagatcatatcatgatcaagacatctaaaggcattcattttcgactatattcttttttacaaaaaatataacaaccagatattttaagctgatcctagatgcacaaaaaataaataaaagtataaacctacttcgtaggatacttcggggtactttttgttcggggttagatgagcataacgcttgtagttgatatttgagatcccctatcattgcagggtgacagcggagcggcttcgcagagctgcattaaccagggcttcgggcaggccaaaaactacggcacgctccggccacccagtccgccggaggactccggttcagggagcggccaactagccgagaacctcacctatgcctggcacaatatggacatctttggggcggtcaatcagccgggctccggatggcggcagctggtcaaccggacacgcggactattctgcaacgagcgacacataccggcgcccaggaaacatttgctcaagaacggtgagtttctattcgcagtcggctgatctgtgtgaaatcttaataaagggtccaattaccaatttgaaactcagtttgcggcgtggcctatccgggcgaacttttggccgtgatgggcagttccggtgccggaaagacgaccctgctgaatgcccttgcctttcgatcgccgcagggcatccaagtatcgccatccgggatgcgactgctcaatggccaacctgtggacgccaaggagatgcaggccaggtgcgcctatgtccagcaggatgacctctttatcggctccctaacggccagggaacacctgattttccaagccatggtgcggatgccacgacatctgacctatcggcagcgagtggcccgcgtggatcaggtgatccaggagctttcgctcagcaaatgtcagcacacgatcatcggtgtgcccggcagggtgaaaggtctgtccggcggagaaaggaagcgtctggcattcgcctccgaggctctaaccgatccgccgcttctgatctgcgatgagcccacctccggactggactcctttaccgcccacagcgtcgtccaggtgctgaagaagctgtcgcagaagggcaagaccgtcatcctgaccattcatcagccgtcttccgagctgtttgagctctttgacaagatccttctgatggccgagggcagggtagctttcttgggcactcccagcgaagccgtcgacttcttttcctagtgagttcgatgtgtttattaagggtatctagtattacataacatctcaactcctatccagcgtgggtgcccagtgtcctaccaactacaatccggcggacttttacgtacaggtgttggccgttgtgcccggacgggagatcgagtcccgtgatcggatcgccaagatatgcgacaattttgccattagcaaagtagcccgggatatggagcagttgttggccaccaaaaatctggagaagccactggagcagccggagaatgggtacacctacaaggccacctggttcatgcagttccgggcggtcctgtggcgatcctggctgtcggtgctcaaggaaccactcctcgtaaaagtgcgacttattcagacaacggtgagtggttccagtggaaacaaatgatataacgcttacaattcttggaaacaaattcgctagattttagatagaattgcctgattccacacccttcttagtttttttcaatgagatgtatagtttatagttttgcagaagataaataaatttcatttaactcgcgaatattaatgagatgcgagtaacattttaatttgcagatggttgccatcttgattggcctcatctttttgggccaacaactcacgcaagtgggtgtgatgaatatcaacggagccatcttcctcttcctgaccaacatgacctttcaaaacgtctttgccacgataaatgtaagtcatgtttagaatacatttgcatttcaataatttactaactttctaatgaatcgattcgatttaggtgttcacctcagagctgccagtttttatgagggaggcccgaagtcgactttatcgctgtgacacatactttctgggcaaaacgattgccgaattgccgctttttctcacagtgccactggtcttcacggcgattgcctatccgatgatcggactgcgggccggagtgctgcacttcttcaactgcctggcgctggtcactctggtggccaatgtgtcaacgtccttcggatatctaatatcctgcgccagctcctcgacctcgatggcgctgtctgtgggtccgccggttatcataccattcctgctctttggcggcttcttcttgaactcgggctcggtgccagtatacctcaaatggttgtcgtacctctcatggttccgttacgccaacgagggtctgctgattaaccaatgggcggacgtggagccgggcgaaattagctgcacatcgtcgaacaccacgtgccccagttcgggcaaggtcatcctggagacgcttaacttctccgccgccgatctgccgctggactacgtgggtctggccattctcatcgtgagcttccgggtgctcgcatatctggctctaagacttcgggcccgacgcaaggagtagccgacatatatccgaaataactgcttgttttttttttttaccattattaccatcgtgtttactgtttattgccccctcaaaaagctaatgtaattatatttgtgccaataaaaacaagatatgacctatagaatacaagtatttccccttcgaacatccccacaagtagactttggatttgtcttctaaccaaaagacttacacacctgcataccttacatcaaaaactcgtttatcgctacataaaacaccgggatatattttttatatacatacttttcaaatcgcgcgccctcttcataattcacctccaccacaccacgtttcgtagttgctctttcgctgtctcccacccgctctccgcaacacattcaccttttgttcgacgaccttggagcgactgtcgttagttccgcgcgattcggttcgctcaaatggttccgagtggttcatttcgtctcaatagaaattagtaataaatatttgtatgtacaatttatttgctccaatatatttgtatatatttccctcacagctatatttattctaatttaatattatgactttttaaggtaattttttgtgacctgttcggagtgattagcgttacaatttgaactgaaagtgacatccagtgtttgttccttgtgtagatgcatctcaaaaaaatggtgggcataatagtgttgtttatatatatcaaaaataacaactataataataagaatacatttaatttagaaaatgcttggatttcactggaactaggctagcataacttcgtataatgtatgctatacgaagttatgctagcggatccgggaattgggaattcacgtaagtactgtctgcagcgtaagcttcgtacgtagcgaccgtctcaaagtactgcctttctgcgttggaaaacatcgcctttttcgtccaaaaggagtccccaggttcgatccgcatggcgttgtgcgtgcgtgcctttcttttcaaatgattacggctattaacttgggggcgttaagttggaaacacgtaaattgcagactgcgattagagtgaccatgagtaggagttcaaaatctcctgacatcattttcttaaaacctgctttgttttttacatttctatttaatataactcctatttgaataaaaaaacaaaacaagtttagatgttaagatattaactacatcctttgctccaaagggagaggggaagttatggagttaattaatttgctgttggaaatcaatatggagtcagaaatataatgatttactaaaccttattgaatcggtaacgatgcgaatttatattaaaatagcttttatgaaacattcaacaaaaatattattaatgttggcccactttagcaaccggttaggtctaccggttgggcaagcaaagattcacgccctggttcgagtcccaactagtcctgcaaaataccgcagcaagttttagagagaccaagtgccattacctctcccacttcagttatcggttatgcggcgtttaagtcgacagcttgccgtctctagctccggtgcctatataaagcagcccgctttccacatttcatattcgttttacgtttgtcaagcctcatagccggcagttcgaacgtatacgctctctgagtcagacctcgaaatcgtagctctacacaattctgtgaattttccttgtcgcgtgtgaaacacttccaaggcaatcttacaaaatgaaactcgccccgtatatcctggagttgctgacgtcggttaataggacaaacggaaccgcagatttgctcgtccccttgttgagggagctcgccaaaggccgtccggttagcaggacgaccttggcaggaatactggattggccagcggagcgcgttgctgcggtcctggagcaagccacgagtaccgagtatgacaaggacggcaatatcatcggatacggtttgacgctgcgcgagactagttatgtctttgaaattgatgacaggcgcttgtacgcatggtgcgctctcgacactctcattttccccgctctgataggacgaactgcaagggtatcgtcccactgcgcagctactggagcccccgtctccctcactgtttccccatccgaaattcaggctgtggaaccagccggtatggccgtgtcgctcgtgctccctcaggaagctgctgacgttcgtcaatccttttgttgtcacgtgcacttttttgcttccgtcccgaccgcagaagactgggcatccaagcatcagggcctggagggcctcgctatagtaagtgtgcatgaagcgtttggattgggtcaggagtttaaccggcacttgctgcaaacaatgagcagtaggacaccttaagcccgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgcccaaactcatcaatgtatcttagcggctcgagggtacctctagagatccactagtgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacactagttctagccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttcctccgaaaagtgccacctaaattgtaagcgttaatatttttgttaaaattcgcgttaaattttttgttaaatcagctcatttttttaaccaataggccgaaatcggcaaaaatccccttataaatcaaaaagaatagaccgagatagggttgagtgttgttcccagttttggc

SEQ ID NO: 5 - DNA, 10368 bp, synthetic construct
  source 1..10368: mol_type = other DNA
aaaataatcaattaggggttccgcgcaacaatttccccgaaaaaagtgcccacctaaattgtaaagcgtttaatatttttgtttaaaattcgcgttaaattttttgttaaatcagctcatttttttaaccaataggccgaaaatcggcaaaaatcccttataaatcaaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacgtaccgggcccctagtatgtatgtaagttaataaaacccatttttgcggaaagtagataaaaaaaacattttttttttttactgcactggatatcattgaacttatctgatcagttttaaatttacttcgatccaagggtatttgatgtaccaggttctttcgattacctctcactcaaaatgacattccactcaaagtcagcgctgtttgcctccttctctgtccacagaaatatcgccgtctctttcgccgctgcgtccgctatctctttcgccaccgtttgtagcgttacgtagcgtcaatgtccgccttcagttgcattttgtcagcggtttcgtgacgaagctccaagcggtttacgccatcaattaaacacaaagtgctgtgccaaaactcctctcgcttcttatttttgtttgttttttgagtgattggggtggtgattggttttgggtgggtaagcaggggaaagtgtgaaaaatcccggcaatgggccaagaggatcaggagctattaattcgcggaggcagcaaacacccatctgccgagcatctgaacaatgtgagtagtacatgtgcatacatcttaagttcacttgatctataggaactgcgattgcaacatcaaattgtctgcggcgtgagaactgcgacccacaaaaatcccaaaccgcaattgcacaaacaaatagtgacacgaaacagattattctggtagctgttctcgctatataagacaatttttgagatcatatcatgatcaagacatctaaaggcattcattttcgactatattcttttttacaaaaaatataacaaccagatattttaagctgatcctagatgcacaaaaaataaataaaagtataaacctacttcgtaggatacttcggggtactttttgttcggggttagatgagcataacgcttgtagttgatatttgagatcccctatcattgcagggtgacagcggagcggcttcgcagagctgcattaaccagggcttcgggcaggccaaaaactacggcacgctccggccacccagtccgccggaggactccggttcagggagcggccaactagccgagaacctcacctatgcctggcacaatatggacatctttggggcggtcaatcagccgggctccggatggcggcagctggtcaaccggacacgcggactattctgcaacgagcgacacataccggcgcccaggaaacatttgctcaagaacggtgagtttctattcgcagtcggctgatctgtgtgaaatcttaataaagggtccaattaccaatttgaaactcagtttgcggcgtggcctatccgggcgaacttttggccgtgatgggcagttccggtgccggaaagacgaccctgctgaatgcccttgcctttcgatcgccgcagggcatccaagtatcgccatccgggatgcgactgctcaatggccaacctgtggacgccaaggagatgcaggccaggtgcgcctatgtccagcaggatgacctctttatcggctccctaacggccagggaacacctgattttccaagccatggtgcggatgccacgacatctgacctatcggcagcgagtggcccgcgtggatcaggtgatccaggagctttcgctcagcaaatgtcagcacacgatcatcggtgtgcccggcagggtgaaaggtctgtccggcggagaaaggaagcgtctggcattcgcctccgaggctctaaccgatccgccgcttctgatctgcgatgagcccacctccggactggactcctttaccgcccacagcgtcgtccaggtgctgaagaagctgtcgcagaagggcaagaccgtcatcctgaccattcatcagccgtcttccgagctgtttgagctctttgacaagatccttctgatggccgagggcagggtagctttcttgggcactcccagcgaagccgtcgacttcttttcctagtgagttcgatgtgtttattaagggtatctagtattacataacatctcaactcctatccagcgtgggtgcccagtgtcctaccaactacaatccggcggacttttacgtacaggtgttggccgttgtgcccggacgggagatcgagtcccgtgatcggatcgccaagatatgcgacaattttgccattagcaaagtagcccgggatatggagcagttgttggccaccaaaaatctggagaagccactggagcagccggagaatgggtacacctacaaggccacctggttcatgcagttccgggcggtcctgtggcgatcctggctgtcggtgctcaaggaaccactcctcgtaaaagtgcgacttattcagacaacggtgagtggttccagtggaaacaaatgatataacgcttacaattcttggaaacaaattcgctagattttagatagaattgcctgattccacacccttcttagtttttttcaatgagatgtatagtttatagttttgcagaagataaataaatttcatttaactcgcgaatattaatgagatgcgagtaacattttaatttgcagatggttgccatcttgattggcctcatctttttgggccaacaactcacgcaagtgggtgtgatgaatatcaacggagccatcttcctcttcctgaccaacatgacctttcaaaacgtctttgccacgataaatgtaagtcatgtttagaatacatttgcatttcaataatttactaactttctaatgaatcgattcgatttaggtgttcacctcagagctgccagtttttatgagggaggcccgaagtcgactttatcgctgtgacacatactttctgggcaaaacgattgccgaattgccgctttttctcacagtgccactggtcttcacggcgattgcctatccgatgatcggactgcgggccggagtgctgcacttcttcaactgcctggcgctggtcactctggtggccaatgtgtcaacgtccttcggatatctaatatcctgcgccagctcctcgacctcgatggcgctgtctgtgggtccgccggttatcataccattcctgctctttggcggcttcttcttgaactcgggctcggtgccagtatacctcaaatggttgtcgtacctctcatggttccgttacgccaacgagggtctgctgattaaccaatgggcggacgtggagccgggcgaaattagctgcacatcgtcgaacaccacgtgccccagttcgggcaaggtcatcctggagacgcttaacttctccgccgccgatctgccgctggactacgtgggtctggccattctcatcgtgagcttccgggtgctcgcatatctggctctaagacttcgggcccgacgcaaggagtagccgacatatatccgaaataactgcttgttttttttttttaccattattaccatcgtgtttactgtttattgccccctcaaaaagctaatgtaattatatttgtgccaataaaaacaagatatgacctatagaatacaagtatttccccttcgaacatccccacaagtagactttggatttgtcttctaaccaaaagacttacacacctgcataccttacatcaaaaactcgtttatcgctacataaaacaccgggatatattttttatatacatacttttcaaatcgcgcgccctcttcataattcacctccaccacaccacgtttcgtagttgctctttcgctgtctcccacccgctctccgcaacacattcaccttttgttcgacgaccttggagcgactgtcgttagttccgcgcgattcggttcgctcaaatggttccgagtggttcatttcgtctcaatagaaattagtaataaatatttgtatgtacaatttatttgctccaatatatttgtatatatttccctcacagctatatttattctaatttaatattatgactttttaaggtaattttttgtgacctgttcggagtgattagcgttacaatttgaactgaaagtgacatccagtgtttgttccttgtgtagatgcatctcaaaaaaatggtgggcataatagtgttgtttatatatatcaaaaataacaactataataataagaatacatttaatttagaaaatgcttggatttcactggaactaggctagcataacttcgtataatgtatgctatacgaagttatgctagcggatccgggaattgggaattcacgtaagtactgtctgcagcgtaagcttcgtacgtagcgaccgtctcaaagtactgcctttctgcgttggaaaacatcgcctttttcgtccaaaaggagtccccaggttcgatccgcatggcgttgtgcgtgcgtgcctttcttttcaaatgattacggctattaacttgggggcgttaagttggaaacacgtaaattgcagactgcgattagagtgaccatgagtaggagttcaaaatctcctgacatcattttcttaaaacctgctttgttttttacatttctatttaatataactcctatttgaataaaaaaacaaaacaagtttagatgttaagatattaactacatcctttgctccaaagggagaggggaagttatggagttaattaatttgctgttggaaatcaatatggagtcagaaatataatgatttactaaaccttattgaatcggtaacgatgcgaatttatattaaaatagcttttatgaaacattcaacaaaaatattattaatgttggcccactttagcaaccggttaggtctaccggttgggcaagcaaagattcacgccctggttcgagtcccaactagtcctgcaaaataccgcagcaagttttagagagaccaagtgccattacctctcccacttcagttatcggttatgcggcgtttaagtcgacagcttgccgtctctagctccggtgcctatataaagcagcccgctttccacatttcatattcgttttacgtttgtcaagcctcatagccggcagttcgaacgtatacgctctctgagtcagacctcgaaatcgtagctctacacaattctgtgaattttccttgtcgcgtgtgaaacacttccaaggcaatcttacaaaatgactcaaaacagctataaaatccctattcagggtatgacctgtactggctgtgaggagcatgtgactgaggctctcgagcaggcgggtgcgaaggatgtaagcgcagactttaggcggggcgaggcgatcttcgaactgagcgatgaccaaatcgaaaaggctaagcaaaatatctccgccgctggataccaacctggtgaagaagaaagtcagccctccgaaaactccgtagactttaatcgcgatggtgactatgacttgttgatcataggctcgggaggagcagccttttccgctgcaattaaggccaatgaaaacggcgcgaaggttgccatggttgaacgcggcacggtgggcggcacctgcgtaaatataggatgcgtacctagcaaaacaatgttgcgcgcaggcgagatcaatggactcgctcagaataacccctttacaggtttgcagaccagcacaggtgctgccgacctggcgcagctcactgaacagaaagacggtctggtatcccagatgaggcaggagaagtacatagatttgattgaggaatatggattcgacctgattcgcggcgaagcaagcttcattgacgacaagaccatccaggtaaacggccagaacattactagtaagtcctttctgatagctacgggcgcaagtcccgcggtccctgagattcctggtatgaatgaagttgattacttgacttcgacctcggccctcgagctgaaagaagtacctcaacgcctcgccgttataggaagcggatacatagctgcggaattgggacagatgtttcataatttgggcactgaggttactctcatgcagcgaagtgagcggctgtttaagacatacgaccctgagataagtgaagcaatagacgagagtctcactgagcaaggtctgaacctcataaccggtgttacgtaccagaaggtggaacagaatggcaaaagtactagcatttatatcgaggtcaatggccaagaacaagtaattgaggcggaccaggttctcgtggcaaccggtcggaagccaaatactgagacgctcaacctcgagagtgctggagtgaagacgggcaaaaaaggtgaagttttgacaaacgaatacttgcaaacaagtaataatcgcatttatgcggcaggcgatgtaacgctcggcccacagtttgtctacgttgcagcctacgaaggtggtatcgtggcgaacaatgctttgggcttggcaaagcgtaagatagacttgcgatttgtgccgggtgtgacctttactaacccaagcatagcgacagtgggcctcacagagcagcaagccaaagaaaaaggatatgatgtcaaaacgagtgtcctcccgctcgacgccgtacccagggcgctcgttaaccacgaaacgacgggcgtttacaagttggttgttaatgctcagacacaaaaattgatcggcgctcatatagtgagcgagaatgcgggagatgtgatttatgcagccacactggcggtgcagttcggactcaccatagaggatctcaccgatagtttcgcgccgtatctcaccatggccgaaggtttgaaactggcggctctcacatttgataaggatgtctcgaagctctcgtgttgcgcaggctaagcccgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgcccaaactcatcaatgtatcttagcggctcgagggtacctctagagatccactagtgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacactagttctagccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttcctccgaaaagtgccacctaaattgtaagcgttaatatttttgttaaaattcgcgttaaattttttgttaaatcagctcatttttttaaccaataggccgaaatcggcaaaaatccccttataaatcaaaaagaatagaccgagatagggttgagtgttgttcccagttttggc

SEQ ID NO: 6 - DNA, 9375 bp, synthetic construct
  source 1..9375: mol_type = other DNA
aaaataatcaattaggggttccgcgcaacaatttccccgaaaaaagtgcccacctaaattgtaaagcgtttaatatttttgtttaaaattcgcgttaaattttttgttaaatcagctcatttttttaaccaataggccgaaaatcggcaaaaatcccttataaatcaaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacgtaccgggcccctagtatgtatgtaagttaataaaacccatttttgcggaaagtagataaaaaaaacattttttttttttactgcactggatatcattgaacttatctgatcagttttaaatttacttcgatccaagggtatttgatgtaccaggttctttcgattacctctcactcaaaatgacattccactcaaagtcagcgctgtttgcctccttctctgtccacagaaatatcgccgtctctttcgccgctgcgtccgctatctctttcgccaccgtttgtagcgttacgtagcgtcaatgtccgccttcagttgcattttgtcagcggtttcgtgacgaagctccaagcggtttacgccatcaattaaacacaaagtgctgtgccaaaactcctctcgcttcttatttttgtttgttttttgagtgattggggtggtgattggttttgggtgggtaagcaggggaaagtgtgaaaaatcccggcaatgggccaagaggatcaggagctattaattcgcggaggcagcaaacacccatctgccgagcatctgaacaatgtgagtagtacatgtgcatacatcttaagttcacttgatctataggaactgcgattgcaacatcaaattgtctgcggcgtgagaactgcgacccacaaaaatcccaaaccgcaattgcacaaacaaatagtgacacgaaacagattattctggtagctgttctcgctatataagacaatttttgagatcatatcatgatcaagacatctaaaggcattcattttcgactatattcttttttacaaaaaatataacaaccagatattttaagctgatcctagatgcacaaaaaataaataaaagtataaacctacttcgtaggatacttcggggtactttttgttcggggttagatgagcataacgcttgtagttgatatttgagatcccctatcattgcagggtgacagcggagcggcttcgcagagctgcattaaccagggcttcgggcaggccaaaaactacggcacgctccggccacccagtccgccggaggactccggttcagggagcggccaactagccgagaacctcacctatgcctggcacaatatggacatctttggggcggtcaatcagccgggctccggatggcggcagctggtcaaccggacacgcggactattctgcaacgagcgacacataccggcgcccaggaaacatttgctcaagaacggtgagtttctattcgcagtcggctgatctgtgtgaaatcttaataaagggtccaattaccaatttgaaactcagtttgcggcgtggcctatccgggcgaacttttggccgtgatgggcagttccggtgccggaaagacgaccctgctgaatgcccttgcctttcgatcgccgcagggcatccaagtatcgccatccgggatgcgactgctcaatggccaacctgtggacgccaaggagatgcaggccaggtgcgcctatgtccagcaggatgacctctttatcggctccctaacggccagggaacacctgattttccaagccatggtgcggatgccacgacatctgacctatcggcagcgagtggcccgcgtggatcaggtgatccaggagctttcgctcagcaaatgtcagcacacgatcatcggtgtgcccggcagggtgaaaggtctgtccggcggagaaaggaagcgtctggcattcgcctccgaggctctaaccgatccgccgcttctgatctgcgatgagcccacctccggactggactcctttaccgcccacagcgtcgtccaggtgctgaagaagctgtcgcagaagggcaagaccgtcatcctgaccattcatcagccgtcttccgagctgtttgagctctttgacaagatccttctgatggccgagggcagggtagctttcttgggcactcccagcgaagccgtcgacttcttttcctagtgagttcgatgtgtttattaagggtatctagtattacataacatctcaactcctatccagcgtgggtgcccagtgtcctaccaactacaatccggcggacttttacgtacaggtgttggccgttgtgcccggacgggagatcgagtcccgtgatcggatcgccaagatatgcgacaattttgccattagcaaagtagcccgggatatggagcagttgttggccaccaaaaatctggagaagccactggagcagccggagaatgggtacacctacaaggccacctggttcatgcagttccgggcggtcctgtggcgatcctggctgtcggtgctcaaggaaccactcctcgtaaaagtgcgacttattcagacaacggtgagtggttccagtggaaacaaatgatataacgcttacaattcttggaaacaaattcgctagattttagatagaattgcctgattccacacccttcttagtttttttcaatgagatgtatagtttatagttttgcagaagataaataaatttcatttaactcgcgaatattaatgagatgcgagtaacattttaatttgcagatggttgccatcttgattggcctcatctttttgggccaacaactcacgcaagtgggtgtgatgaatatcaacggagccatcttcctcttcctgaccaacatgacctttcaaaacgtctttgccacgataaatgtaagtcatgtttagaatacatttgcatttcaataatttactaactttctaatgaatcgattcgatttaggtgttcacctcagagctgccagtttttatgagggaggcccgaagtcgactttatcgctgtgacacatactttctgggcaaaacgattgccgaattgccgctttttctcacagtgccactggtcttcacggcgattgcctatccgatgatcggactgcgggccggagtgctgcacttcttcaactgcctggcgctggtcactctggtggccaatgtgtcaacgtccttcggatatctaatatcctgcgccagctcctcgacctcgatggcgctgtctgtgggtccgccggttatcataccattcctgctctttggcggcttcttcttgaactcgggctcggtgccagtatacctcaaatggttgtcgtacctctcatggttccgttacgccaacgagggtctgctgattaaccaatgggcggacgtggagccgggcgaaattagctgcacatcgtcgaacaccacgtgccccagttcgggcaaggtcatcctggagacgcttaacttctccgccgccgatctgccgctggactacgtgggtctggccattctcatcgtgagcttccgggtgctcgcatatctggctctaagacttcgggcccgacgcaaggagtagccgacatatatccgaaataactgcttgttttttttttttaccattattaccatcgtgtttactgtttattgccccctcaaaaagctaatgtaattatatttgtgccaataaaaacaagatatgacctatagaatacaagtatttccccttcgaacatccccacaagtagactttggatttgtcttctaaccaaaagacttacacacctgcataccttacatcaaaaactcgtttatcgctacataaaacaccgggatatattttttatatacatacttttcaaatcgcgcgccctcttcataattcacctccaccacaccacgtttcgtagttgctctttcgctgtctcccacccgctctccgcaacacattcaccttttgttcgacgaccttggagcgactgtcgttagttccgcgcgattcggttcgctcaaatggttccgagtggttcatttcgtctcaatagaaattagtaataaatatttgtatgtacaatttatttgctccaatatatttgtatatatttccctcacagctatatttattctaatttaatattatgactttttaaggtaattttttgtgacctgttcggagtgattagcgttacaatttgaactgaaagtgacatccagtgtttgttccttgtgtagatgcatctcaaaaaaatggtgggcataatagtgttgtttatatatatcaaaaataacaactataataataagaatacatttaatttagaaaatgcttggatttcactggaactaggctagcataacttcgtataatgtatgctatacgaagttatgctagcggatccgggaattgggaattcacgtaagtactgtctgcagcgtaagcttcgtacgtagcgaccgtctcaaagtactgcctttctgcgttggaaaacatcgcctttttcgtccaaaaggagtccccaggttcgatccgcatggcgttgtgcgtgcgtgcctttcttttcaaatgattacggctattaacttgggggcgttaagttggaaacacgtaaattgcagactgcgattagagtgaccatgagtaggagttcaaaatctcctgacatcattttcttaaaacctgctttgttttttacatttctatttaatataactcctatttgaataaaaaaacaaaacaagtttagatgttaagatattaactacatcctttgctccaaagggagaggggaagttatggagttaattaatttgctgttggaaatcaatatggagtcagaaatataatgatttactaaaccttattgaatcggtaacgatgcgaatttatattaaaatagcttttatgaaacattcaacaaaaatattattaatgttggcccactttagcaaccggttaggtctaccggttgggcaagcaaagattcacgccctggttcgagtcccaactagtcctgcaaaataccgcagcaagttttagagagaccaagtgccattacctctcccacttcagttatcggttatgcggcgtttaagtcgacagcttgccgtctctagctccggtgcctatataaagcagcccgctttccacatttcatattcgttttacgtttgtcaagcctcatagccggcagttcgaacgtatacgctctctgagtcagacctcgaaatcgtagctctacacaattctgtgaattttccttgtcgcgtgtgaaacacttccaaggcaatcttacaaaatgaaaaatatctcggagttcagtgctcagttggatcaaacgtttgaccaaggagaagcagttagcatggagtggctctttaggcccctcctcaagatgttggcggaaggagaccccgtgcccgtcgaggatatagcagcagagaccggtaaacctgtagaggaagtgaagcaagtattgcagacgctcccatcggtggaactcgacgaacaaggtcgtgtcgttggctatggcctgacgctgttccctacgcctcatcgttttgaagtcgatggtaaacagttgtatgcgtggtgtgcgctggacacgctgatgtttccagctttgattggacgtacggtccatatcgcgtccccatgtcacggtaccggcaagagcgtaaggctcactgttgagccggaccgtgtcgtatcggttgaaccgagtacggctgtggtgtcgattgtaacacccgatgaaatggcgtccgttcgcagtgcgttctgtaatgacgtacacttttttagcagccccagcgcggcgcaggattggctcaaccagcaccccgagtcctcggtcctgccggtggaagacgcatttgaactcggtcgtcatttgggtgctaggtacgaagagagcggtccgactaacggtagttgttgtaacatataagcccgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgcccaaactcatcaatgtatcttagcggctcgagggtacctctagagatccactagtgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacactagttctagccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttcctccgaaaagtgccacctaaattgtaagcgttaatatttttgttaaaattcgcgttaaattttttgttaaatcagctcatttttttaaccaataggccgaaatcggcaaaaatccccttataaatcaaaaagaatagaccgagatagggttgagtgttgttcccagttttggc

SEQ ID NO: 7 - DNA, 639 bp, synthetic construct
  source 1..639: mol_type = other DNA
atgaaactcgccccgtatatcctggagttgctgacgtcggttaataggacaaacggaaccgcagatttgctcgtccccttgttgagggagctcgccaaaggccgtccggttagcaggacgaccttggcaggaatactggattggccagcggagcgcgttgctgcggtcctggagcaagccacgagtaccgagtatgacaaggacggcaatatcatcggatacggtttgacgctgcgcgagactagttatgtctttgaaattgatgacaggcgcttgtacgcatggtgcgctctcgacactctcattttccccgctctgataggacgaactgcaagggtatcgtcccactgcgcagctactggagcccccgtctccctcactgtttccccatccgaaattcaggctgtggaaccagccggtatggccgtgtcgctcgtgctccctcaggaagctgctgacgttcgtcaatccttttgttgtcacgtgcacttttttgcttccgtcccgaccgcagaagactgggcatccaagcatcagggcctggagggcctcgctatagtaagtgtgcatgaagcgtttggattgggtcaggagtttaaccggcacttgctgcaaacaatgagcagtaggacaccttaa

SEQ ID NO: 8 - DNA, 1695 bp, synthetic construct
  source 1..1695: mol_type = other DNA
atgtcgaccctcaagatcactggtatgacctgcgatagctgtgcagtccacgtaaaggatgcgctcgaaaaggtcccgggtgtccaatccgctgacgtgtcctacgcaaaaggttccgctaagctggcaattgaagtaggaacgtcgcccgatgcactcactgccgccgtagcaggactgggatatagggcgacactcgccgacgcagcatcggtctcgactcctggaggtctgttggataaaatgcgggacctgttgggtcgtaacgacaaaactggaagcagtggagctctgcatatagcagttattggttcgggtggcgcagcaatggccgcggccctgaaggccgtggagcaaggcgcacgggtcactctgattgaacgaggtacgatcggtggaacttgcgtgaatgtaggttgcgtcccttccaaaataatgattcgtgcagcacatatagcgcatctccgtcgtgaatcgccctttgacggcggaattgctgccacgactcccaccatccagcgaacagcactgctcgcccagcaacaagcgagggtagatgagctcaggcacgcaaagtacgaaggcattctcgagggcaaccctgccattacagttttgcatggcagtgcacgcttcaaggacaaccgtaacctgattgtgcagctgaatgacggcggcgaacgagtcgtggccttcgacaggtgcctcatcgccaccggagcgtcccccgcagtgccgccaatacctggtttgaaagacaccccctattggacatccacagaagccttggttagtgaaacaattccgaagcgcctggcagtaattggtagctcggttgtagctctcgaattggcgcaggcgtttgcgcgactgggagcgaaggtaacaattttggcccgcagtaccttgttttttcgagaagacccagctattggcgaggcggtcacagctgccttccggatggagggtatcgaggttcgagagcacactcaggcatcccaggtagcctatatcaatggcgagggtgatggcgagttcgtcctgaccaccgctcatggcgagctccgggccgataagctgctggttgcgacaggccgagcgccaaatacccgcaagctggcattggatgcaacaggcgtaacgttgacgcctcaaggagcgatcgttatcgatccgggtatgcgcacatccgtcgagcacatttatgctgcaggcgattgtacggaccagcctcaattcgtctatgttgccgcggccgctggtacacgcgctgccataaacatgactggtggcgacgctgcgttgaatctcacagcgatgcccgccgtggtgttcacagacccccaggtcgctacagtcggttatagcgaagccgaagctcatcacgacggcatcaagaccgacagccgcacattgaccctcgataatgtccctagggcgctcgctaatttcgatacgcgtggattcattaaactcgtagtcgaagaaggaagcggtcgcttgatcggcgtgcaagcagttgctccagaagcgggtgagctcattcaaacggccgcactcgccattcggaatcgcatgacagtacaggagctggctgatcaactgtttccctatctcacgatggtagagggcctcaagctcgccgcacagacgttcaacaaagatgtgaagcaactcagttgctgtgccggctaa